(19) United States
(12) Patent Application Publication
(10) Pub. No.: US 2009/0010906 A1
(43) Pub. Date: Jan. 8, 2009

(54) PERORAL DOSAGE FORMS TO ACHIEVE A SUSTAINED-RELEASE EFFECT AFTER MEDICAMENT DOSAGE WITH A MEAL

(76) Inventors: Henning Blume, Bad Homburg (DE); Ernst Mutschler, Mainz (DE); Werner Weitschies, Neuenkirchen (DE)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/574,317

(22) PCT Filed: Aug. 30, 2005
(86) PCT No.: PCT/EP2005/054260

§ 371 Date: Dec. 28, 2007

(30) Foreign Application Priority Data
Aug. 31, 2004 (DE) 10 2004 042 139.0

(51) Int. Cl. A61K 38/43; A61K 31/702; A61K 31/435

(52) U.S. Cl. 424/94.1; 514/61; 514/338

(57) ABSTRACT

The present invention relates a retard formulation for oral administration together with a meal comprising at least one active pharmaceutical ingredient and optionally one or more pharmaceutically acceptable excipient(s) as well as an amount of an agent being able to form a gas (hereinafter also referred to as gas forming agent or gas former), which gas forming agent achieves a homogeneous mixture of the active pharmaceutical ingredient, hereinafter referred to as API, with the content of the stomach, hence providing a continuous initial absorption phase of the API.

The present invention relates a retard formulation for oral administration together with a meal comprising at least one active pharmaceutical ingredient and optionally one or more pharmaceutically acceptable excipient(s) as well as an amount of an agent being able to form a gas (hereinafter also referred to as gas forming agent, gas former or gas developer), which gas forming agent achieves a homogeneous mixture of the Active Pharmaceutical Ingredient, hereinafter referred to as API, with the content of the stomach, hence providing a continuous initial absorption phase of the API.

When administering pharmaceutical formulations via the oral route comprising an API with immediate release behavior together with a meal, very often delays regarding the initial absorption of the API appear, which usually becomes apparent from the fact situation, that—as compared to application in the fasting state—the maximum systemic concentration of the API is lower while the time until the maximum concentration of the API is reached is prolonged. Oral administration of retard formulations together with or after a meal show—as compared to application in the fasting state—often a prolonged time until first systemic concentrations of the API suspect to measurement can be observed. In some cases one can even describe that significant plasma concentrations appear even only several hours after administration of the retard formulation, which effect is also described as “dose dumping”, see for example Schug B S, Brendel E, Wonnemann M, Wolf D, Wargenau M, Dingier A, Blume H H, “dosage form-related food interaction observed in a marketed once-daily nifedipine formulation after a high-fat American breakfast” in Eur J. Clin. Pharmacol. 2002; 58(2): 119-125.

According to the prior art it is known to use gas forming agents in effervescent formulations. Such effervescent formulations are solved in aqueous media prior to administration and the gas forming agent only acts in such a way that the dosage form quickly decomposes in the aqueous medium. Hence, the gas development occurs already prior to administration of the API, namely during decomposition of the formulation in a suited amount of water (see, for example, Bauer K H, Frömming K H, Fuhrer C; Pharmazeutische Technologie, 5^{th} edition, Gustav Fischer Verlag, Stuttgart 1997, p. 314).

One should note that after administration of such a solved effervescence a continuous initial absorption and systemic circulation of the API cannot be achieved. Rather, the known effervescent formulations which are administered orally lead to a particularly quick initial absorption of the API which occurs after ingestion of the effervescent formulation being decomposed in a suited liquid, which is in many cases water.

The use of gas forming agents as excipients in solid oral dosage forms casually takes place for production of tablets or chewing tablets suited for oral administration, with the gas forming agent acting as an disintegration accelerator, for example disclosed in “Oral tablet disintegrant of nimodipine for treating dementia”, Faming Zhuanli Shenqing Gongkai Shuomingshu, 6 pp. CODEN: CNXXEV; Chinese; CN 1394605. In such cases development of gas occurs during the administration in the mouth or also in the stomach, however, for such formulations the incorporated amount of gas developing (or gas forming) agent is too small to prevent a discontinuous increase of the respective active pharmaceutical ingredient.

DE 691 25 619 T2 refers to structures for achieving controlled release, which controlled release can be achieved by coating a longish extrudated core with a material being insoluble in water. Calcium carbonate is mentioned as suitable excipient, however, not in connection with any controlled release behavior. Rather, the presence of calcium carbonate does not lead to a retarding effect but causes a quicker release, as apparent from the figures of this patent document.

Therefore, it is the object of the present invention to provide dosage forms which can significantly diminish the problem of undesired high plasma levels (plasma peaks) as well as delays with initial absorption of APIs administered orally together with or after a meal. Furthermore, the dosage forms should as much as possible achieve a continuous concentration curve of the API within the systemic circulation.

Surprisingly it has been found that the use of gas forming agents as excipients in solid oral dosage forms significantly diminishes undesired peaks of API plasma levels as well as delays with the initial absorption of APIs upon oral administration together with or after a meal. This is assumedly based on the fact that the amount of gas developing agent(s) being present in the dosage forms according to the invention allow a preferably homogeneous admixing of the also present API(s) (as well as other parts of the dosage form comprising APIs) with the content of the stomach.

Upon administration of a dosage form according to the invention after or during a meal, also for APIs being properly soluble in water, over a period of at least 30 minutes, usually for more than one hour, a continuous initial absorption of the one or more API(s) being present in the formulation within the systemic circulation can be observed. This retarding effect is surprising and contrary to the up-to-date usual practice to use gas forming agents as excipients within oral solid dosage forms, since known effervescent dosage forms should assure a particular quick release of the API. In the context of retarding dosage forms, up-to-date gas developers (used to accelerate decomposition) have not been used, since with such agents the problem of dose dumping in connection with administration together with a meal is expected to be even worse. As opposed to this knowledge around gas developers as pharmaceutical excipients in solid oral dosage forms, the dosage forms according to the present invention are characterized in that the generation of gas occurs only after ingestion within the stomach of a patient, allowing a continuous initial absorption phase of the API.

One prerequisite for the advantageous effect of the dosage form according to the invention is the presence of an adequate amount of gas forming agent in the dosage form and another one is the administration of the drug during or preferably after a meal. The dosage form according to the invention preferably is to be administered while within the stomach sugars, carbohydrates, fats and/or proteins are present.

According an embodiment of the present invention, for prolonging or improvement of the retarding effect, the API present in the dosage form may completely or partly be in a controlled or slow release form. Also gastro-resistant forms are suited wherein the API can be protected from the low pH of the gastric juice. The person skilled in the art will, varying from API to API, take into consideration retarding formulations and/or gastro-resistant coatings.

In this context, for example, wrapped particles or particles consisting of a matrix of excipients, such as granulate particles or pellets or also microparticles, can be suited. For embodiments comprising the API in particulate form together with excipients, the particle size of the single particles is less than 2 mm, preferably less than 1 mm.

Administration of the dosage forms according to the invention irrespective of food intake, that means probably even in a fasting state, is not dangerous for the patent at all, since the API can also be effective if administered in such a manner.

As gas developing agent compounds releasing carbon dioxide (CO₂) are suited. Preferred are sodium hydrogen carbonate, sodium carbonate, calcium carbonate and magnesium carbonate or mixtures thereof. One may mix two or more of the mentioned gas developing agents in any ratio. The person skilled in the art will choose a respective API or a combination of APIs as a suited composition of gas developing agents, depending on the agent or combination of agents. As mentioned before, also further chemical entities may be chosen, provided that they release carbon dioxide.

For the dosage forms according to the invention such an amount of gas developing agents is used for each single dosage that makes a vast homogeneous mixture of the API with the content of the stomach possible. For example 50 mg of a gas developing agent may be a suited amount per single dosage. According to a preferred embodiment at least 100 to 150 mg of a gas developing agent per administration are administered, more preferred at least 250 mg, further preferred 300 mg, and if reasonable 500 mg and even up to 1,000 mg of gas developing agent per administration. The skilled person will, depending on API and particular galenic formulation, choose the amount of gas developing agent necessary and determine the same such that the effect according to the present invention of homogeneously admixing the AIP with the content of the stomach is achieved.

Acid which is necessary for preferred gas developing agents according to the invention for releasing carbon dioxide may origin from the content of the stomach or, alternatively, be present in the oral dosage form. Suited for admixing are all physiologically acceptable acids with excipients like citric acid, tartaric acid, ascorbic acid or mixtures thereof being preferred. In particular preferred is ascorbic acid. The one or more acid according to preferred embodiments of the present invention are present in amounts of 100 to 300 mg preferably of 100 to 200 mg, particular preferred in an amount of about 200 mg in the oral dosage form according to the invention. The person skilled in the art will choose a suited amount of acid depending on the oral dosage form.

The oral dosage forms according to the present invention are in particular suited for the following systemic effective APIs: Tricylic antidepressants, such as Amitryptiline, Doxepine and Imipramine; non-steroidal antiphlosgistics, such as in particular Indomethacine, Diclofenac and Ketoprofene; analgetics, like Oxycodone, Orphine, Tramadol and Tilidin; antiepileptica, such as Carbamacepine, Oxcarbacepine, valproinic acid, Phenyloin and Gabapentin; antiparkinson agents, such as Levodopa and Enthacapone; alpha-receptor-blocking agents, such as Doxazosine; betablocking agents, such as Bisoprolole, Atenolole and Metoprolole; spasmolytics such as Oxybutynine; anti-dementiva, such as Menantine and Donepizile; tyroid hormones, such as Levothyroxine and Liothyronine; calcium-antagonists, such as Felodipine, Nifedipine, Nitrendipine, Amlodipine and Diltiazem; protone pump inhibitors (PPIs), such as Omeprazol, Pantoprazol and Lansoprazol; chinolones such as Norfloxacine, Ofloxacine, Ciprofloxacine, Levofloxacine and Moxifloxacine; loop diuretics such as Furosemide and Torasemide; oral antidiabetics, such as Metformin, Glibenclamide, Glimepidride, Repaglinide and Nateglinide as well as nicotinic acid and pentaerythrityltetranitrate.

Also after administration of dosage forms comprising active ingredients being effective mostly pre-systemically in the intestine or during the first passage of the intestine wall or the liver, a preferably homogeneous mixture of the API with a content of the stomach is advantageous. For that reason the dosage form according to the invention are also in particular suited for administration of the APIs Acarbose, Miglitole, pancreatic enzymes, Ezetemibe, statines, such as Atorvastatin, Fluvastatin, Lovastatin, Pravastatin, Simvastatin, as well as Orlistat. In particular suited is the API Acarbose.

Dosage forms according to the invention can be coated or non-coated tablets, chew tablets, capsules, coated or non-coated granules, coated or non-coated powders or suspensions. The necessary dosage of active ingredient per single dosage can be divided into more than one dosage form, i.e. can be divided into several individually separated dosage forms as, for example, two or three tablets or capsules. The necessary amount of gas forming agent relates in such a scenario to the total amount of administered active pharmaceutical ingredient.

The following examples describe the dosage forms according to the invention without limitation:

### EXAMPLE 1

Tablet consisting of 500 mg sodium hydrogen carbonate, 100 mg Acarbose, 100 mg microcrystalline cellulose, and 10 mg magnesium stearate. Sodium hydrogen carbonate, Acarbose and microcrystalline cellulose are mixed in a tumbling mixer, magnesium stearate is added and short mixing is repeated. The resulting powder is pressed into a tablet making use of a tablet press.

### EXAMPLE 2

Tablet consisting of 400 mg sodium hydrogen carbonate, 100 mg citric acid, 50 mg Acarbose, 100 mg microcrystalline cellulose, 10 mg magnesium stearate. Sodium hydrogen carbonate, citric acid, Acarbose and microcrystalline cellulose are mixed in a free fall mixer, magnesium stearate is added and again a short mixture takes place. The resulting powder is pressed into a tablet making use of a tablet press.

### EXAMPLE 3

Capsule comprising 250 mg sodium hydrogen carbonate, 25 mg Acarbose, 5 mg magnesium stearate. Sodium hydrogen carbonate und Acarbose are mixed in a free fall mixer, magnesium stearate is added and again a short mixture takes place. The powder is filled into a hard gelatine capsule.

### EXAMPLE 4

Capsule comprising 250 mg sodium hydrogen carbonate, 50 mg citric acid, 25 mg Acarbose, 5 mg magnesium stearate. Sodium hydrogen carbonate, citric acid and Acarbose are mixed in a free fall mixer, magnesium stearate is added and again a short mixture takes place. The powder is filled into a hard gelatin capsule.

### EXAMPLE 5

Capsule comprising 10 mg Omeprazol in the form of pellets in gastric juice resistant form (Omeprazol, Stada), 300 mg sodium hydrogen carbonate, 50 mg ascorbic acid, 5 mg magnesium stearate. Sodium hydrogen carbonate and acid are mixed in a free fall mixer, magnesium stearate and the gastric juice resistant pellets (Omeprazol, Stada) are added. Again a mixture takes place. The mixture is filled into a hard gelatin capsule.

### EXAMPLE 6

Capsule comprising 10 mg Omeprazol in the form of pellets in gastric juice resistant form (Omeprazol, Stada), 300 mg sodium hydrogen carbonate, 50 mg ascorbic acid, 50 mg microcrystalline cellulose, 5 mg magnesium stearate. Sodium hydrogen carbonate, acid and microcrystalline cellulose are mixed in a free fall mixer. After addition of magnesium stearate the powder is compacted and thereafter is crashed into granules. The granulate and the gastric juice resistant pellets (Omeprazol, Stada) are admixed. The mixture is filled into a hard gelatin capsule.

### EXAMPLE 7

50 mg Acarbose, 406 mg ascorbic acid and 194 mg sodium hydrogen carbonate are mixed in a mortar and filled into a hard gelatine capsule (seize 1).

During a randomized clinical study 5 healthy test persons received in the morning in the fasting state after one half of a defined breakfast (oat flakes (100 g), milk (100 ml, 1.5% fat) and saccharose (50 g) a hard gelatine capsule according to the invention or alternatively as a reference a conventional quick release tablet comprising 50 mg Acarbose (sold under the brand Glucobay®).

Between the two alternative applications at least 7 days of elutriation time was held.

Blood samples (1.5 ml each) were gained at the following points in time: 5 min prior to begin of the breakfast and then 10 min, 15 min, 20 min, 25 min, 30 min, 35 min, 40 min, 45 min, 50 min, 55 min, 60 min, 1 h 10 min, 1 h 20 min, 1 h 30 min, 1 h 40 min, 1 h 50 min, 2 h, 2 h 15 min, 2 h 30 min, 2 h 45 min, 3 h, 3 h 15 min, 3 h 30 min, 3 h 45 min, 4 h, 4 h 20 min, 4 h 40 min, 5 h, 5 h 20 min, 5 h 40 min, 6 h, 6 h 20 min, 6 h 40 min, 7 h, 7 h 20 min, 7 h 40 min, 8 h after begin of the breakfast. The content of glucose of the blood samples was determined.

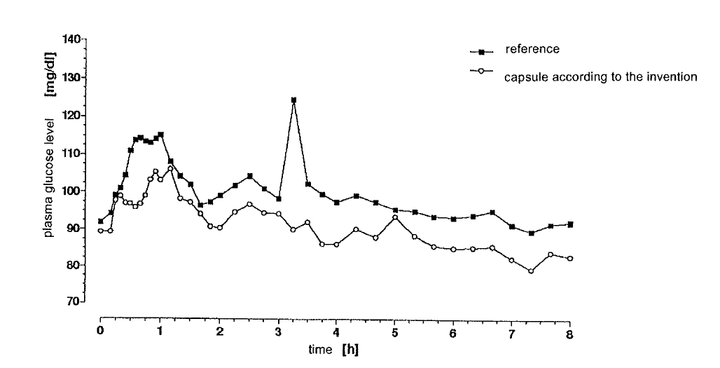

For the amount of glucose in the blood the result shown in FIG. 1 was obtained. It can be seen that application of the dosage form according to the invention (here: hard gelatine capsules) the glucose level in the plasma was advantageous, namely the Acarbose shows a continuous and long lasting effect while undesired peak concentrations can be avoided.

1. Retard formulation for oral administration together with a meal, comprising at least an Active Pharmaceutical Ingredient (API) and optionally one or more pharmaceutically acceptable excipients as well as an amount of a gas forming agent, which allows a widely homogeneous mixing of the API with the content of the stomach thus allowing a continuous initial absorption phase of the API.

2. Retard formulation according to claim 1 comprising per application dosage at least 50 mg of the gas forming agent.

3. Retard formulation according to claim 1 and/or 2, comprising per application dosage at least 150 mg of the gas forming agent.

4. Retard formulation according to one or more of claims 1 to 3, wherein the gas forming agent is selected from sodium hydrogen carbonate, sodium carbonate, calcium carbonate and magnesium carbonate or mixtures thereof.

5. Retard formulation according to one or more of claims 1 to 4, wherein the API is selected from one or more out of the group consisting of tricyclic antidepressants, non steroidal antiphlogistics, analgetics, antiepileptics, alpha receptor blocking agents, beta blocking agents, spasmolytics, antidementiva, thyroid hormones, proton pump inhibitors (PPIs), chinolones, loop diuretics or oral antidiabetics.

6. Retard formulation according to one or more of claims 1 to 5, wherein the API is selected from Acarbose, Miglitol, pankreatic enzymes, Ezetemibe, Statins, such as Atorvastatine, Fluvastatine, Lovastatine, Pravastatine, Simvastatine, or Orlistat.

7. Use of a gas forming agent for the manufacture of a medicament for administration of an API together with a meal while simultaneously achieving a retarding effect.

8. Use according to claim 7, wherein the gas forming agent is a carbonate.

9. Use according to claim 7 and/or 8, wherein the gas forming agent is selected from sodium hydrogen carbonate, sodium carbonate, calcium carbonate and magnesium carbonate or mixtures thereof.

10. Use according to one or more of claims 7 to 9, wherein the Active Pharmaceutical Ingredient is selected from one or more out if the group consisting of tricyclic antidepressants, non steroidal antiphlogistics, analgetics, antiepileptics, alpha receptor blocking agents, beta blocking agents, spasmolytics, antidementiva, thyroid hormones, proton pump inhibitors (PPIs), chinolones, loop diuretics or oral antidiabetics.

11. Use according to one or more of claims 7 to 10, wherein the Active Pharmaceutical Ingredient is selected from Acarbose, Miglitol, pancreatic enzymes, Ezetemibe, statines, such as Atorvastatine, Fluvastatine, Lovastatine, Pravastatine, Simvastatine, or Orlistat.

12. Use according to one or more of claims 7 to 11, wherein the gas forming agent is present in an amount of at least 50 mg per single application dosage.

13. Use according to one or more of claims 7 to 12, wherein the gas forming agent is present in an amount of at least 150 mg per single application dosage.
